(19) United States
(12) Patent Application Publication
(10) Pub. No.: US 2023/0062599 A1
(43) Pub. Date: Mar. 2, 2023

(54) DEVICES, SYSTEMS, AND METHODS FOR ANCHORING AN ARTIFICAL CHORDAE TENDINEAE TO CARDIAC TISSUE

(71) Applicant: Boston Scientific Scimed, Inc., Maple Grove, MN (US)

(72) Inventors: Joseph Walker, Shoreview, MN (US); Troy A. Giese, Blaine, MN (US); Christopher J. Koudela, New London, MN (US); Daniel Shuey, Pine City, MN (US); Aaron Abbott, Columbia Heights, MN (US); Joel T. Eggert, Plymouth, MN (US); Larry M. Killeen, Elk River, MN (US); James P. Rohl, Prescott, WI (US); Mitchell Nelson, Hudson, WI (US)

(73) Assignee: BOSTON SCIENTIFIC SCIMED, INC., Maple Grove, MN (US)

(21) Appl. No.: 17/899,585

(22) Filed: Aug. 30, 2022

Related U.S. Application Data
(60) Provisional application No. 63/239,469, filed on Sep. 1, 2021.

(51) Int. Cl. A61F 2/24

(52) U.S. Cl. CPC A61F 2/2466; A61F 2/2457; A61F 2220/0016; A61F 2210/0014

(57) ABSTRACT

A tissue anchor and an anchor delivery and deployment system. The tissue anchor is shiftable between a delivery configuration when housed in anchor garage of the anchor delivery and deployment system, and a deployment configuration when deployed outside the anchor garage. The anchor has a plurality of talons which may be formed from a laser cut tube. The talons may taper, such as widthwise.

### CROSS-REFERENCE TO RELATED APPLICATIONS

This application claims the benefit of priority of U.S. Provisional Application No. 63/239,469, filed Sep. 1, 2021, the entire disclosure of which is hereby incorporated by reference herein for all purposes.

### FIELD

The present disclosure relates generally to the field of implantable medical devices. In particular, the present disclosure relates to medical devices, systems, and methods for cardiac treatment. More particularly, the present disclosure relates to medical devices, systems, and methods for delivering and deploying an anchor such as for anchoring an artificial chordae tendineae to heart tissue.

### BACKGROUND

Heart disease, including atrioventricular heart valve malfunctions, impedes patient cardiac output, which reduces patient quality of life and lifespan. The proper flow of blood through the heart is regulated, inter alia, by heart valves, including atrioventricular heart valves, which include soft tissue leaflets which cyclically open and close to allow blood to flow through in one direction. Healthy leaflets prevent blood flow in the opposite direction (regurgitation). Chordae tendineae, extending from the leaflets to the papillary muscles, support the proper functioning of the leaflets, such as by distributing load to the papillary muscles during systolic closure, and by preventing the leaflet from flailing into the atrium. Improper functioning of the chordae tendineae compromises the capacity of the leaflets to form a seal at the heart valve. Various defects of failure of the chordae tendineae, such as elongation, rupture, thickening, retraction, calcification, inelastic stretching or other changes in elasticity, etc., may result in improper closure of the heart valve and/or a flailing leaflet that may no longer have the capacity to form a valving seal for normal heart function.

Heart valve disease is typically repaired via invasive surgical intervention or by complicated pinching of the leaflets together creating dual, smaller openings, or a replacement of the native valve. These approaches involve risky by-pass surgery that may include an opening into the patient's chest and heart chamber to expose the heart valve for direct viewing and repair. Resection, partial removal, and/or repair of the patient's leaflets along with the implantation of a surgical ring are complex techniques used by surgeons to reduce the diameter of the patient's heart valve annulus, thus allowing the leaflets to properly coapt and reduce regurgitate flow. Some techniques may slightly reduce regurgitate flow but may not provide a durable solution and do not repair and/or replace damaged chordae tendineae of a valve. Thus, transluminal solutions to valve disease are needed.

There is a need for minimally invasive solutions to repair a heart valve, such as the leaflets thereof, while maintaining the option for future interventions. Moreover, there is a need for improvements to tissue anchors and to devices, systems, and methods for delivering and deploying a tissue anchor, and/or securing the anchor to heart tissue.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an anchor delivery and deployment system includes an anchor having an anchor body and a plurality of anchor talons extending from the anchor body and shiftable between a delivery configuration and a deployed configuration, and an anchor garage configured to house the anchor therein with the anchor talons in a delivery configuration. The anchor garage has an open blunt end through which the anchor is deployable. The anchor talons expand from the delivery configuration when within the anchor garage to the deployment configuration when deployed outside the anchor garage.

In some embodiments, the blunt end of the anchor garage has a convex curved outer surface to present a blunt end for pressing against tissue without damaging the tissue.

In some embodiments, the anchor talons extend from a talon base coupled to the anchor body to a talon distal end configured to penetrate tissue, and at least one of the talons is tapered between the talon base and the talon distal end. In some embodiments, at least one of the talons is tapered toward the talon distal end. In some embodiments, at least one of the talons is tapered from the talon base to the talon distal end. Additionally or alternatively, at least one of the talons has an intermediate region between the talon base and the talon distal end. In some embodiments, the at least one of the talons is tapered from the intermediate region to the talon distal end. Additionally or alternatively, the at least one of the talons is tapered from the intermediate region to the talon base.

In some embodiments, at least one of the talons is tapered widthwise.

In some embodiments, the anchor is formed from a tube, and the talons are cut from the tube. In some embodiments, the talons are formed by laser cutting the tube from which the anchor body is formed.

In some embodiments, the system further comprises an artificial chordae tendineae coupled to the anchor.

In accordance with various principles of the present disclosure, a tissue anchor includes an anchor body formed from a tube, and a plurality of anchor talons formed by cutting the tube. The anchor talons extend from a talon base coupled to the anchor body to a talon distal end configured to penetrate tissue, and at least one of the talons is tapered between the talon base and the talon distal end.

In some embodiments, at least one of the talons is tapered toward the talon distal end.

In some embodiments, at least one of the talons is tapered widthwise.

In some embodiments, the anchor talons extend from a talon base coupled to the anchor body to a talon distal end configured to penetrate tissue, and at least one of the talons is tapered between the talon base and the talon distal end. In some embodiments, at least one of the talons is tapered toward the talon distal end. In some embodiments, at least one of the talons is tapered from the talon base to the talon distal end. Additionally or alternatively, at least one of the talons has an intermediate region between the talon base and the talon distal end. In some embodiments, the at least one of the talons is tapered from the intermediate region to the talon distal end. Additionally or alternatively, the at least one of the talons is tapered from the intermediate region to the talon base.

In some embodiments, the talons are formed by laser cutting the tube from which the anchor body is formed.

In accordance with various principles of the present disclosure, a method of securing an anchor in tissue includes delivering a tissue anchor in an anchor garage, the anchor garage having an open blunt distal end, the tissue anchor having a plurality of talons each extending from a talon base to a talon distal end; pressing the blunt distal end of the anchor garage against tissue without damaging the tissue; advancing the anchor talon distal ends out of the blunt distal end of the anchor garage and to penetrate tissue; and advancing the anchor out of the anchor garage to deploy the anchor in tissue, allowing the anchor talons to shift from a delivery configuration to a deployed configuration within the tissue.

In some embodiments, the anchor and anchor talons are formed from a laser cut tube.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements of illustrated anchors are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of an anchor delivery and deployment device and system formed in accordance with various principles of the present disclosure shown in a schematic representation of a heart for deployment in cardiac tissue.

FIG. 2 illustrates a perspective view of an example of an embodiment of an anchor delivery and deployment device and system formed in accordance with various principles of the present disclosure, and with a delivery catheter and leaflet clip delivery/deployment system optionally associated therewith illustrated in phantom.

FIG. 3 illustrates a cross-sectional view along line of an example of an embodiment of an anchor delivery and deployment device and system as in FIG. 2 with an elevational view of an anchor formed in accordance with various principles of the present disclosure shown within the anchor delivery and deployment system in a delivery configuration.

FIG. 4 illustrates a cross-sectional view along line IV-IV of an example of an embodiment of an anchor delivery and deployment device and system as in FIG. 2 with an elevational view of an anchor formed in accordance with various principles of the present disclosure shown within the anchor delivery and deployment system in a deployed configuration.

FIG. 5A illustrates a perspective view of an example of an embodiment of an anchor formed in accordance with various principles of the present disclosure.

FIG. 5B illustrates a planar projection of the example of an embodiment of an anchor of FIG. 5A.

FIG. 6A illustrates a perspective view of an example of an embodiment of an anchor formed in accordance with various principles of the present disclosure.

FIG. 6B illustrates a planar projection of the example of an embodiment of a cardiac anchor of FIG. 6A.

FIG. 7A illustrates a perspective view of an example of an embodiment of an anchor formed in accordance with various principles of the present disclosure.

FIG. 7B illustrates a planar projection of the example of an embodiment of a cardiac anchor of FIG. 7A.

FIG. 8A illustrates a perspective view of an example of an embodiment of a cardiac anchor formed in accordance with various principles of the present disclosure.

FIG. 8B illustrates a planar projection of the example of an embodiment of a cardiac anchor of FIG. 8A.

FIG. 9A illustrates a perspective view of an example of an embodiment of a cardiac anchor formed in accordance with various principles of the present disclosure.

FIG. 9B illustrates a planar projection of the example of an embodiment of a cardiac anchor of FIG. 9A.

FIG. 10A illustrates a perspective view of an example of an embodiment of a cardiac anchor formed in accordance with various principles of the present disclosure.

FIG. 10B illustrates a planar projection of the example of an embodiment of a cardiac anchor of FIG. 10A.

FIG. 11A illustrates a perspective view of an example of an embodiment of a cardiac anchor formed in accordance with various principles of the present disclosure.

FIG. 11B illustrates a planar projection of the example of an embodiment of a cardiac anchor of FIG. 11A.

FIG. 12 illustrates a schematic representation of a heart with an example of an embodiment of a cardiac anchor system formed in accordance with various principles of the present disclosure anchoring an anchor thereof to a heart.

FIG. 13 illustrates a schematic representation of a heart with an example of an embodiment of a cardiac anchor formed in accordance with various principles of the present disclosure anchored to a heart with an artificial chordae tendineae extending therefrom.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. It will be appreciated that a “bore” is not limited to a circular cross-section. As used herein, a “free end” of an element is a terminal end at which such element does not extend beyond.

Heart disease including atrioventricular heart valve malfunctions impede patient cardiac output, which reduces patient quality of life and lifespan. As heart disease progresses, the chordae tendineae that connect the papillary muscle of the ventricle to a valve leaflet may stretch inelastically and may rupture. A stretched and/or ruptured chordae tendineae may result in a flailing leaflet that may no longer have capacity to form a valving seal for normal heart function. For example, abnormal blood flow regurgitation from the ventricle to the atrium may develop. Regurgitation prevents an adequate supply of blood to be delivered through the cardiovascular systems.

Repositioning, repair, and/or replacement of one or more leaflets of a valve and/or chordae tendinea may be desirable to treat heart disease. The devices, systems, and methods of the present disclosure may be used alone or together with other devices, systems, and methods to treat heart disease. Examples of devices, systems, and methods with which embodiments of the present disclosure may be implemented include, but are not limited to, those described in Does U.S. Patent Application Publication US2021/0007847, titled Devices, Systems, And Methods For Clamping A Leaflet Of A Heart Valve, and published on Jan. 14, 2021; U.S. Patent Application Publication US2021/0000597, titled Devices, Systems, And Methods For Adjustably Tensioning An Artificial Chordae Tendineae Between A Leaflet And A Papillary Muscle Or Heart Wall, and published on Jan. 7, 2021; U.S. Patent Application Publication US2021/0000599, titled Devices, Systems, And Methods For Artificial Chordae Tendineae, and published on Jan. 7, 2021; U.S. Patent Application Publication US2021/0000598, titled Devices, Systems, And Methods For Anchoring An Artificial Chordae Tendineae To A Papillary Muscle Or Heart Wall, and published on Jan. 7, 2021; and U.S. Patent Application Publication US2022/0096235, titled Devices, Systems, And Methods For Adjustably Tensioning Artificial Chordae Tendineae In A Heart, and published on Mar. 31, 2022, each of which is herein incorporated by reference in its entirety and for all purposes. Examples of devices described therein may be modified to incorporate embodiments or one or more features of the present disclosure.

Repositioning, repair, and/or replacement of one or more leaflets of a valve and/or chordae tendinea may include one or more devices to be fixed to one or more leaflets of a heart valve and to cardiac tissue, such as papillary muscle tissue. Examples of embodiments of devices and systems and methods described herein facilitate fixation of one or more devices to heart tissue, such as papillary muscle tissue. Examples of embodiments of devices described herein provide anchoring for other devices, systems, or tools with respect to anatomical structure such as the heart. It will be appreciated that devices and systems described herein may be used with devices or systems disclosed in the above-referenced applications incorporated herein, or may be used with other devices and systems, such as those described herein.

In accordance with various principles of the present disclosure, a tissue anchor, such as for anchoring to cardiac tissue, is disclosed in conjunction with an anchor delivery and deployment system which delivers and deploys the anchor to anatomical tissue such as papillary muscle and/or a heart wall. The anchor delivery and deployment system preferably is configured to be navigated through tortuous pathways within the body for transluminal (in contrast with open surgery) delivery and deployment of the anchor. The anchor delivery and deployment system may be delivered through a delivery catheter or sheath, and optionally other flexible tubular element capable of navigating tortuous pathways within the body. For instance, the anchor delivery and deployment system may be delivered within a delivery catheter configured to deliver a leaflet clip delivery and deployment system, such as described in co-pending patent application [ATTORNEY DOCKET 2001.2700101], titled “Devices, Systems, And Methods For Clamping A Leaflet Of A Heart Valve,” and filed on even date herewith and incorporated by reference herein in its entirety for all purposes.

In accordance with various principles of the present disclosure, an anchor delivery and deployment system includes an anchor garage configured to house the anchor therein during delivery and having a blunt open distal end to be pressed against the tissue as the anchor is ejected from the garage (though the blunt distal end). The blunt end is particularly configured so as not to damage the tissue into which the anchor is secured. It will be appreciated that the term anchor element is used for the sake of convenience and may be used interchangeably herein with terms such as anchor, anchor device, anchor mechanism, anchor component, anchoring element, anchoring device, anchoring mechanism, anchoring component, and the like, such terms being known in the art to represent structures configured to hold another object in place. It will further be appreciated that various terms (in various grammatical forms) may be used interchangeably herein to refer to the attachment of an anchor to tissue, such as attach, anchor, implant, affix, secure, couple, engage, hold, retain, etc., without intent to limit. Finally, terms such as eject, push, deploy, release, propel, expel, dispense, etc., (and various grammatical forms thereof) may be used interchangeably herein without intent to limit.

An anchor delivery and deployment system formed in accordance with various principles of the present disclosure is configured to deliver an anchor disposed within an anchor garage in a delivery configuration. In some embodiments, the anchor garage is disposed within a catheter, typically with an approximately 0.22 in (5.59 mm) inner diameter, and the anchor garage may have an outer diameter of approximately 0.12 in (3.048 mm) outer diameter (and, in some embodiments, an inner diameter of approximately 0.1065 in/2.7051 mm). In some embodiments, the anchor includes a plurality of talons which are biased into a deployed configuration for engaging tissue, yet which may be positioned in a delivery configuration for delivery to tissue, such as within an anchor delivery and deployment device of an anchor delivery and deployment system. The delivery configuration may be a compact or compressed configuration, and the deployment configuration may be an open or expanded configuration (with an outer dimension greater than the outer dimension of the anchor when in a delivery configuration). For instance, the anchor garage may constrain or hold the anchor talons in a compact configuration to fit within the inner diameter of the anchor garage. In some embodiments, the anchor talons are extended or elongated in the compact configuration to extend along a longitudinal axis of the anchor garage. An actuator, such as a pusher rod, may be used to move the anchor out of the anchor garage for deployment in body tissue. Once the anchor is no longer within the anchor delivery and deployment device, the anchor talons may move into the open configuration, such as an expanded configuration. In the expanded configuration, the anchor may have an outer diameter greater than its outer diameter when in the delivery configuration and greater than the inner diameter of the anchor garage.

If pushed against body tissue, the anchor talons may pierce and penetrate into the tissue and spread into a deployed configuration secured within the body tissue. The anchor may be formed of an elastic and/or shape memory material, such as Nitinol, formed (e.g., by heat treating) into the desired open configuration. As such, the anchor talons are biased to substantially automatically open into the open configuration without external forces moving the anchor talons. It will be appreciated that terms such as open, expand, shift, move, transition, etc. (and various grammatical forms thereon) may be used interchangeably herein to refer to the movement of the anchor talons without intent to limit.

In accordance with various principles of the present disclosure, the anchor talons may be configured such that the free ends thereof bend inwardly, away from the walls of the anchor garage, when the anchor is positioned within the anchor garage (e.g., in a delivery configuration). As such, the anchor is slidable or otherwise movable within the anchor garage without the free ends of the talons interfering with, catching on, damaging, or being impeded by the anchor garage. On the other hand, the free ends of the anchor talons may be configured to be able to penetrate (e.g., pierce) into body tissue when the anchor is pushed out of the anchor garage and contacts body tissue. In such embodiments, the angle of the anchor talons with respect to the longitudinal axis of the anchor is not so great as to present a blunt end to the tissue, and, instead, allows the free ends of the talons (particularly, a surface of the free ends generally transverse to the longitudinal axis of the talons/anchor) to be pressed against the tissue into which the anchor is to be deployed so that the talons penetrate the tissue. Selection of such angle of the talons may be balanced with selection of an angle which reduces or prevents the free ends of the talons from moving against the walls of the anchor garage.

The anchor talons may be sized, shaped, configured, and/or dimensioned to ensure engagement with and retention in tissue, yet to permit flexing or bending of the talons between a delivery configuration and a deployed configuration. For instance the thickness, width, length, and other dimensions may be selected to ensure tissue engagement yet to permit flexing of the talons without deforming or otherwise adversely structurally or functionally affecting the anchor talons. Additionally or alternatively, the anchor talons may be sized, shaped, configured, and/or dimensioned so that despite the degree of curvature of the talons to achieve sufficient purchase in tissue, stresses through the talons are sufficiently distributed to avoid deformation or failure (e.g., breaking) of the talons under the range of tensions or forces thereon (e.g., delivery forces, tensioning forces, forces once deployed, etc.). Such forces may be readily appreciated by those of ordinary skill in the art. In some embodiments, the anchor talons are formed with barbs or other features to increase purchase with tissue once deployed.

In some embodiments, the anchor talons are tapered, such as tapered widthwise (e.g., from side to side, or circumferentially about the anchor). For instance, the anchor talons may be tapered from a base thereof (at which the talons extend from the anchor body) to a free end thereof. Alternatively, the anchor talons may be tapered from an intermediate region thereof to the base thereof and to the free ends thereof (i.e., wider at an intermediate region between the base and the free ends).

In some embodiments described herein, an anchor is formed from a laser-cut tube. In such embodiments, the anchor talons may extend from an anchor base which may be in the form of a ring or tube (the remaining portion of the tube from which the talons are cut or formed). The anchor base may also form the anchor body or may be a part of the anchor body.

In other embodiments, the anchor talons may instead be formed separately from an anchor base and coupled thereto, such as by welding, or other manners of affixing. The anchor base may be coupled to an anchor body or to another element associated with the anchor. For instance, in some embodiments, an artificial chordae tendineae tensioning and locking device, configured and structured to receive an artificial chordae tendineae and to hold the artificial chordae tendineae in a desired position and/or configuration, is associated with (e.g., coupled to) the anchor. The anchor base may be coupled to the artificial chordae tendineae tensioning and locking device, such as disclosed in any of the patent applications referenced and/or incorporated by reference herein. Coupling of the anchor base to another element may be via coupling elements, such as mating elements (e.g., a pin extending through holes in the anchor body and anchor base, threading, a detent and groove engagement, etc.), or by crimping or welding, or by any other suitable coupling sufficiently strong to withstand pulling forces on the anchor body by the element coupled thereto (e.g., an artificial chordae tendineae) in a direction away from the anchor talons and anchor base.

As will be appreciated, delivery, deployment, and engagement of the disclosed embodiments may be facilitated by use of known medical visualization techniques, such as fluoroscopy, ultrasound, intra-cardiac echography, or the like.

Various embodiments of tissue anchors and anchor delivery and deployment devices, systems, and methods, will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Finally, it should be appreciated that various dimensions provided herein are examples, and one of ordinary skill in the art can readily determine the standard deviations and appropriate ranges of acceptable variations therefrom (e.g., in view of nominal dimensions, shape, stress/strain considerations, etc.) which are covered by the present disclosure and any claims associated therewith. Therefore, the present disclosure is not limited to only the embodiments specifically described herein.

Turning now to the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

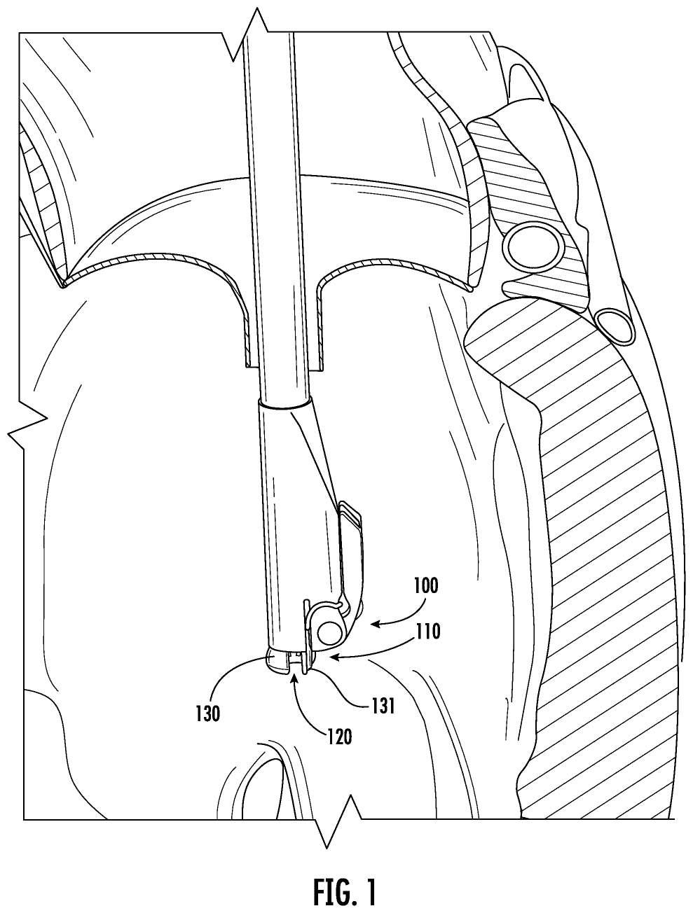

An anchor device and system 100 formed in accordance with various principles of the present disclosure is illustrated in FIG. 1 positioned to deploy a cardiac anchor. The anchor device and system 100 includes an anchor delivery and deployment device 110 configured to deliver and to deploy an anchor 120 carried therein.

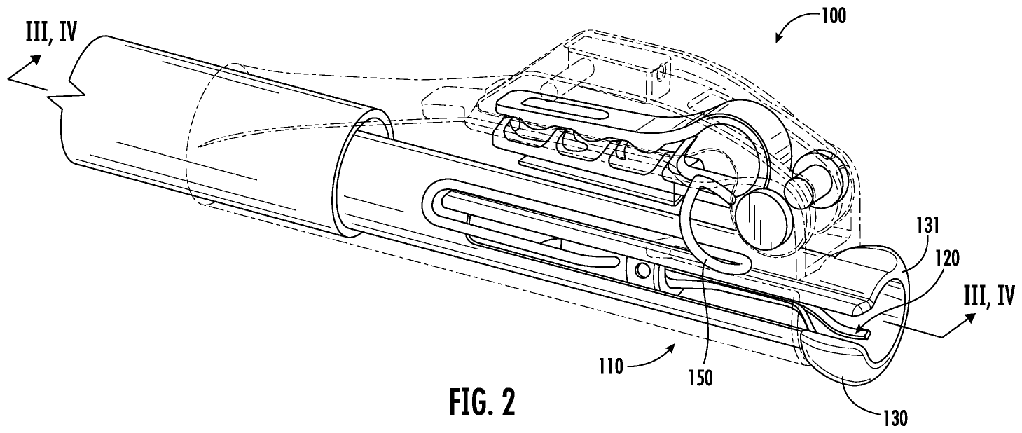

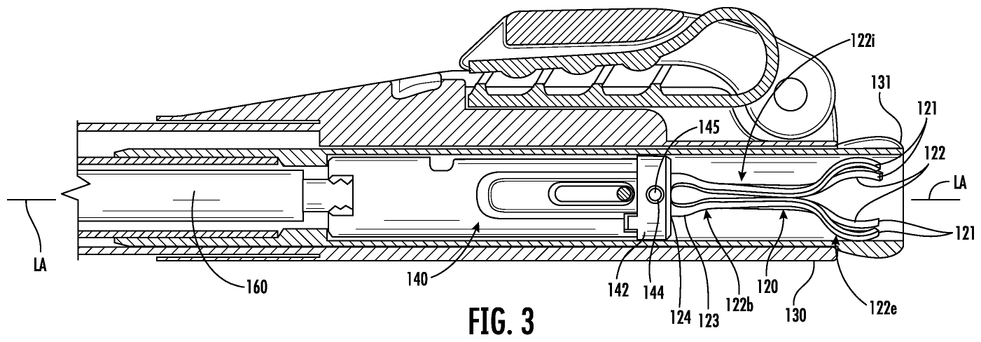

In accordance with various principles of the present disclosure, the anchor device and system 100 includes an anchor garage 130 configured to house or to hold the anchor in a generally unexpanded delivery configuration, as may be appreciated with reference to FIG. 2 and FIG. 3. The anchor garage 130 may alternately be referenced herein as a delivery housing, sheath, etc. without intent to limit. In the example of an embodiment illustrated in FIG. 2, the anchor garage 130 may be configured with an open blunt distal end 131 (tip or free end). The blunt distal end 131 is sized, shaped, configured, and dimensioned to facilitate pushing of the anchor garage 130 against cardiac tissue to deploy the anchor 120 through the open blunt distal end 131 and out of the anchor garage 130 without potentially pushing the distal end 131 of the anchor garage 130 into the cardiac tissue as well. For instance, the distal end 131 of the anchor garage 130 may be radiused or curved inwardly (presenting a convex curved outer surface) or otherwise formed to be sufficiently blunt as to not injure tissue against which the anchor garage 130 is pushed. For example, and without limitation, edges of the distal end 131 of the anchor garage 130 may be curved with a radius of approximately 0.0275 in (0.6985 mm).

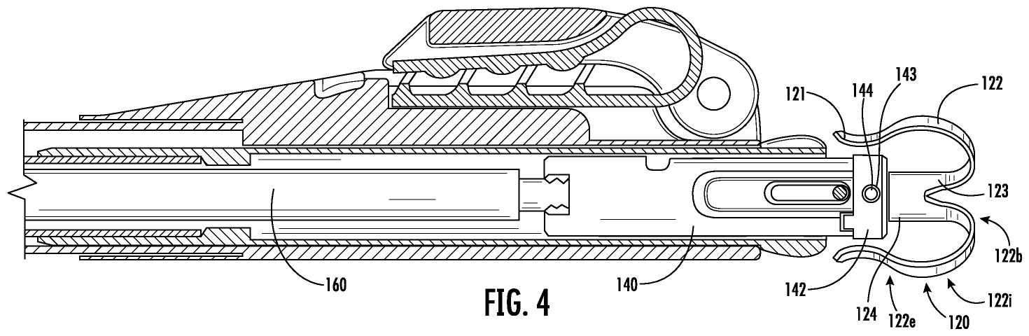

As may be appreciated with reference to FIG. 3, an anchor 120 formed in accordance with various principles of the present disclosure has a plurality of talons 122 extending distally from an anchor body 124 to distal ends 121 thereof. The anchor 120 may be coupled with an artificial chordae tendineae tensioning and locking device 140 to anchor an artificial chordae tendineae 150 (see, e.g., FIG. 2) with the anchor 120. The artificial chordae tendineae tensioning and locking device 140 is structured and configured to receive an artificial chordae tendineae and to hold the artificial chordae tendineae in a desired position and/or configuration, such as with respect to the anchor 120. The specific structure of the artificial chordae tendineae tensioning and locking device 140 is not critical to the present disclosure and may be any desired structure, including, but not limited to, structures disclosed in any of the patent applications referenced and/or incorporated by reference herein. In some embodiments, an end cap 142 is provided to couple the anchor 120 (e.g., via the anchor body 124) and the artificial chordae tendineae tensioning and locking device 140. In the example of an embodiment illustrated in FIG. 3 and FIG. 4, the end cap 142 is interlocked with the anchor body 124 via a locking pin 144 extending through a bore 145 through the end cap 142 and a corresponding bore (not illustrated in FIG. 3, but described in further detail below) within the anchor body 124. A mechanical interference fit may be advantageous if the anchor 120 is formed from nitinol and the artificial chordae tendineae tensioning and locking device is formed from a different material, such as a metal (e.g., medical grade stainless steel) or a polymer. However, it will be appreciated that other manners of coupling the anchor 120 and the artificial chordae tendineae tensioning and locking device 140 are within the scope and spirit of the present disclosure.

In a delivery configuration of the example of an embodiment of an anchor 120 illustrated in FIG. 3, the anchor talons 122 are generally elongated and extend along the longitudinal axis LA of the anchor device and system 100. The talons 122 extend from a talon base 123 coupled with the anchor body 124 to the distal ends 121 thereof. The base region 122b, the intermediate region 122i, and the end region 122e of the talons 122 extend distally away from the anchor body 124.

As may be appreciated with reference to FIG. 4, distal advancement of the anchor 120 relative to the anchor garage 130 allows the anchor 120 to shift into an expanded deployed configuration in which talons 122 of the anchor 120 curve outwardly from the longitudinal axis LA along which the talons 122 generally are positioned when in the delivery configuration illustrated in FIGS. 2 and 3. It will be appreciated that the term curve (and other grammatical forms thereof, such as used with reference to the anchor talons 122, may be used interchangeably herein with terms such as bend, bow, shape, etc. (and other grammatical forms thereof). An anchor pusher rod 160 may be used to advance the anchor 120 distally out of the anchor garage 130 to extend into a deployed configuration, such as illustrated in FIG. 4, such as to be deployed in tissue (as discussed in further detail below). The anchor 120 may be formed from a shape memory material (e.g., Nitinol) which shifts or returns the talons 122 from an extended configuration, as illustrated FIG. 3, to an expanded curved or bent configuration, such as illustrated in FIG. 4. In the expanded configuration of the example of an embodiment of an anchor 120 illustrated in FIG. 4, the base regions 122b of the talons 122 extend from the respective talon bases 123 distally away from the anchor body 124, and the intermediate regions 1221 of the talons 122 are bent back over themselves in a proximal direction towards the anchor body 124 so that the end regions 122e and distal ends 121 of the anchor talons 122 extend towards (optionally beyond) the anchor body 124.

In accordance with various principles of the present disclosure, the talons 122 of an anchor 120 are configured so as to move relative to the anchor garage 130 without the talons 122 or the interior surface of the anchor garage 130 affecting movement of the anchor 120. More particularly, the talons 122 and the anchor garage 130 may be formed so as not to interfere with, catch on, damage, or be impeded by each other. It will be appreciated that because the talons 122 may be configured and formed (e.g., with shape memory material) to extend without external forces (in other words, to automatically shift or move) into a curved and/or expanded configuration, the talons 122 are curved or otherwise biased radially outwardly (i.e., in a direction away from the longitudinal axis LA of the anchor delivery and deployment system 100). As such, the talons 122 may exert a radially outward force on the wall of the anchor garage 130. Moreover, the end regions 122e of the talons 122 may extend radially outwardly further than the base regions 122b of the talons 122 (in other words, occupy a greater circumferential or cross-sectional area within the anchor garage 130). In accordance with various principles of the present disclosure, such as in the example of an embodiment illustrated in FIG. 3, the end regions 122e of the talons present a generally convex surface to the interior surface of the anchor garage 130. Additionally or alternatively, the distal ends 131 of the talons 122 may be directed somewhat inwardly, to minimize or eliminate any risk of the distal ends 131 scraping the interior surface of the anchor garage 130. For instance, the distal end 131 of the talons 122 may be angled or curved inwardly towards the longitudinal axis LA of the anchor delivery and deployment system 100.

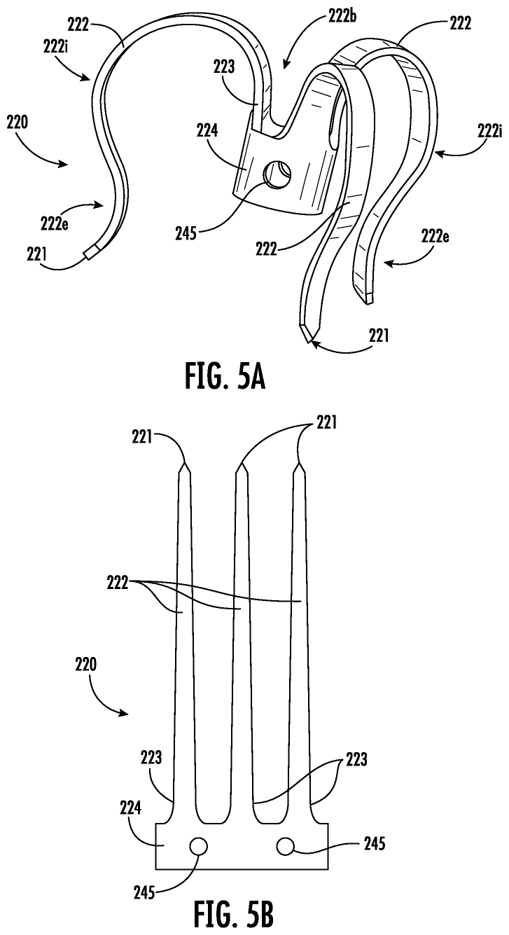

As discussed above, it is generally desirable for an anchor 120 formed in accordance with various principles of the present disclosure to establish a strong purchase in or with the tissue so that the anchor 120 is not inadvertently withdrawn from the tissue. In one aspect of the present disclosure, an artificial chordae tendineae 150 (illustrated in FIG. 2) may be coupled to the anchor 120 and the anchor 120 may be used to anchor an artificial chordae tendineae 150 to cardiac tissue, as discussed in further detail below. In such embodiment, it is generally desirable for the anchor 120 to be dimensioned, shaped, formed, and configured to resist any tension exerted thereon (such as by movement of a leaflet to which the artificial chordae tendineae 150 is also coupled). Various examples of embodiments of anchors formed in accordance with various principles of the present disclosure are illustrated in FIGS. 5A, 5B, 6A, 6B, 7A, 7B, 8A, 8B, 9A, 9B, 10A, 10B, which will now be described in further detail.

Examples of embodiments of anchors 220, 320, 420, 520, 620, 720, 820 formed in accordance with various principles of the present disclosure, and illustrated respectively in FIGS. 5A, 5B, 6A, 6B, 7A, 7B, 8A, 8B, 9A, 9B, 10A, 10B, have a plurality of talons 222, 322, 422, 522, 622, 722, 822, having respective talon bases 223, 323, 423, 523, 623, 723, 823 coupled to a respective anchor body 224, 324, 424, 524, 624, 724, 824, and respective distal ends 221, 321, 421, 521, 621, 721, 821 that extend away from the anchor body 224, 324, 424, 524, 624, 724, 824. The distal ends 221, 321, 421, 521, 621, 721, 821 may each have a tapered or pointed tip suitable for piercing tissue so that the talons 222, 322, 422, 522, 622, 722, 822 may penetrate into tissue and the anchors 220, 320, 420, 520, 620, 720, 820 may be securely fixed to tissue when the anchor is engaged therewith.

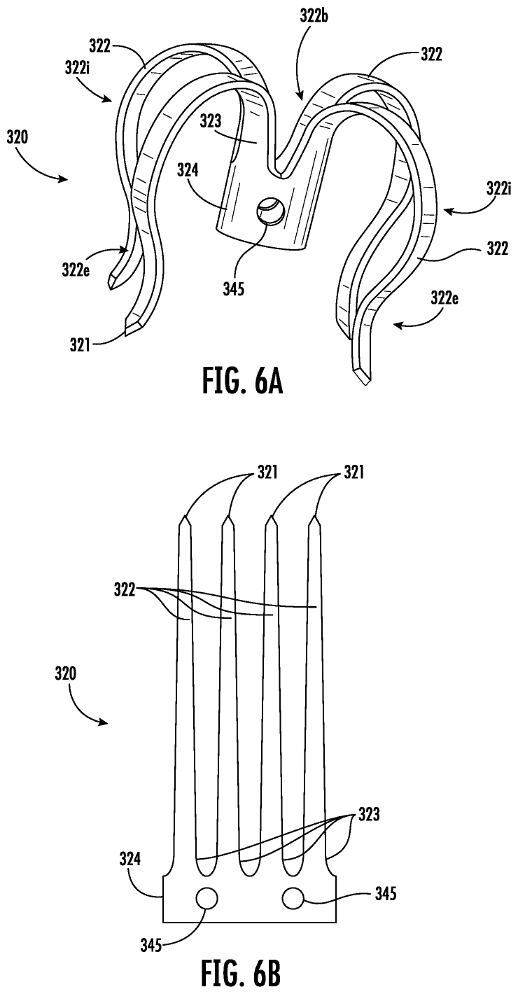

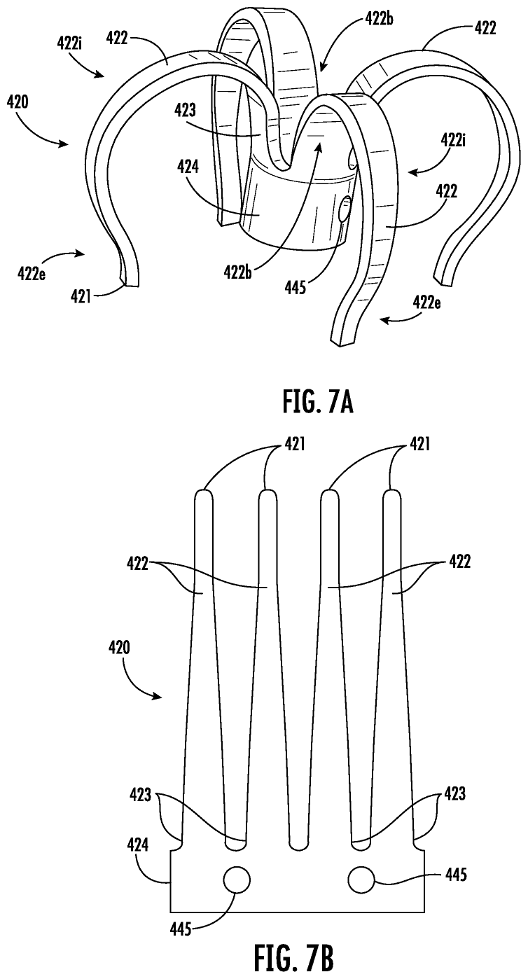

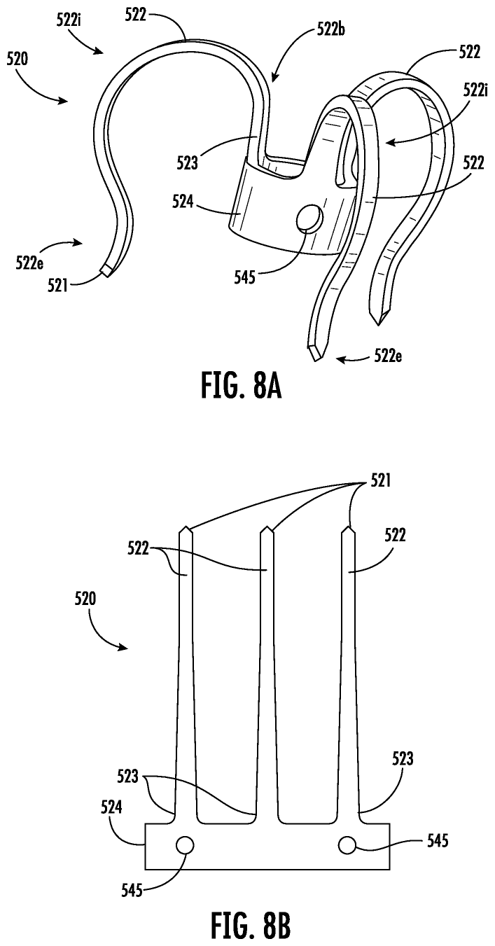

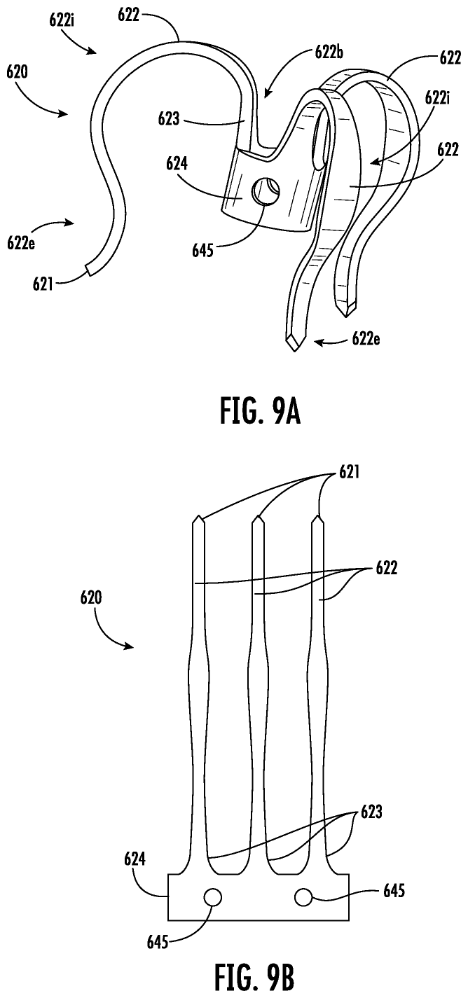

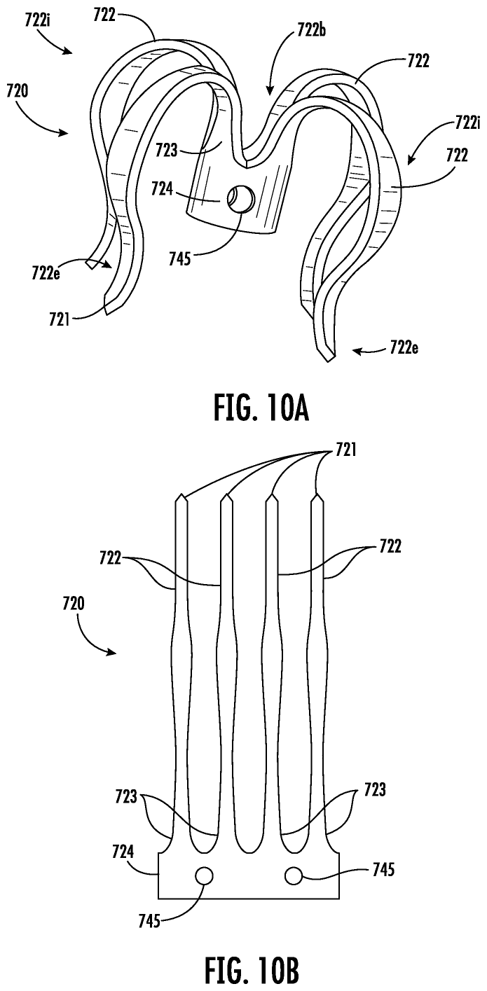

As may be appreciated from the perspective views of FIG. 5A, FIG. 6A, FIG. 7A, FIG. 8A, FIG. 9A, and FIG. 10A, the talons 222, 322, 422, 522, 622, 722, 822 of the illustrated examples of embodiments of anchors 220, 320, 420, 520, 620, 720, 820 may have a bowed appearance such that when the talons 222, 322, 422, 522, 622, 722, 822 are in a neutral position (e.g., not acted upon by an outside force) the respective intermediate regions 222i, 322i, 422i, 522i, 622i, 722i, 822i thereof extend radially outwardly from a respective anchor body 224 324, 424, 524, 624, 724, 824 thereof by a distance greater than a distance between the anchor body 224, 324, 424, 524, 624, 724, 824 and the distal ends 221, 321, 421, 521, 621, 721, 821 of the talons 222, 322, 422, 522, 622, 722, 822. Bowed regions of the talons 222, 322, 422, 522, 622, 722, 822 may act as a spring to move the distal ends 221, 321, 421, 521, 621, 721, 821 of the talons 222, 322, 422, 522, 622, 722, 822 radially outwardly away from the longitudinal axis LA of the anchor delivery and deployment system 100 to bend back towards the anchor body 224, 324, 424, 524, 624, 724, 824 when the anchor 220, 320, 420, 520, 620, 720, 820 is moved from a delivery configuration (e.g., within the anchor garage 130, such as illustrated in FIG. 3) to a deployed configuration (e.g., outside the anchor garage 130, such as illustrated in FIG. 4).

In the deployed configuration of FIG. 3, such as in the perspective views of FIG. 5A, FIG. 6A, FIG. 7A, FIG. 8A, FIG. 9A, and FIG. 10A, the distal ends 221, 321, 421, 521, 621, 721, 821 of each of the talons 222, 322, 422, 522, 622, 722, 822 may point away from the body 224, 324, 424, 524, 624, 724, 824. In such configuration, the distal ends 221, 321, 421, 521, 621, 721, 821 are positioned to engage tissue. In addition, as described above, when the talons 222, 322, 422, 522, 622, 722, 822 are in the extended configuration when the anchor 220, 320, 420, 520, 620, 720, 820 is in a delivery configuration, such curvature may remain so that the distal ends 221, 321, 421, 521, 621, 721, 821 of each of the talons 222, 322, 422, 522, 622, 722, 822 may be directed inwardly away from the interior surface of the anchor garage 130 in which the anchor 220, 320, 420, 520, 620, 720, 820 is delivered.

In accordance with various principles of the present disclosure, an anchor 120, 220, 320, 420, 520, 620, 720, 820 may have two or more talons 222, 322, 422, 522, 622, 722, 822, such as three or four talons as illustrated, for example, in FIG. 3, FIG. 4, FIG. 5A, FIG. 6A, FIG. 7A, FIG. 8A, FIG. 9A, and FIG. 10A. The talons of the anchors may have similar shapes and/or configurations but may be differently proportioned or spaced apart depending on the total number of talons on the anchor upon comparison. For example, the three talons 222 of the example of an embodiment of an anchor 220 illustrated in FIG. 5A and the four talons 322 of the example of an embodiment of an anchor 320 illustrated in FIG. 6A have similar shapes and proportions, but slightly different dimensions and/or relative spacing. More particularly, the talons 322 have smaller dimensions and/or are closer together than the talons 222 so that the anchor 320 can accommodate more talons than accommodated by the anchor 220. Similarly, the three talons 422 of the example of an embodiment of an anchor 420 illustrated in FIG. 7A and the four talons 522 of the example of an embodiment of an anchor 520 illustrated in FIG. 8A have similar shapes and proportions, but slightly different dimensions and/or relative spacing. More particularly, the talons 522 have smaller dimensions and/or are closer together than the talons 422 so that the anchor 520 can accommodate more talons than accommodated by the anchor 620.

In accordance with various principles of the present disclosure, widthwise tapering of the talons (e.g., side to side, or along the perimeter of the anchor) has been found to provide the talons with sufficient flexibility and also sufficient resistance to strain along the talons, such as the base thereof (e.g., at the talon base 223, 323, 623, 723). One or more talons of an anchor such as disclosed herein may be formed in accordance with various principles of the present disclosure to include one or more tapered regions. In the examples of embodiments of anchors 220, 320, 620 illustrated, respectively, in FIG. 5A, FIG. 5B, FIG. 6A, FIG. 6B, FIG. 9A, FIG. 9B, FIG. 10A, FIG. 10B, the respective anchor talons 220, 320, 620, 720 have a single taper, from the talon base 223, 323, 623 723 to the talon distal end 221, 321, 621, 721. However, other configurations are within the scope and spirit of the present disclosure to achieve the desired anchoring in and with respect to tissue, and the desired delivery and deployment configurations. For instance, in the examples of embodiments of anchors 420, 520 illustrated, respectively, in FIG. 7A, FIG. 7B, FIG. 8A, FIG. 8B, the talons 422, 522 have two tapers, from the intermediate region 422i, 422i to the end region 422e, 422e, and from the intermediate region 422i, 522i to the base region 422b, 422b of the respective talons 422, 522. It will be appreciated that talons of anchors formed in accordance with various principles of the present disclosure may additionally or alternatively have regions with generally uniform width, or with additional tapers or other variations in width without departing from the scope and spirit of the present disclosure.

In accordance with various principles of the present disclosure, the degree and location of tapering along the talons of anchors and/or the overall dimension of the anchor may affect the spacing between the talons. For example, a greater degree of tapering, as in the example of an embodiment of an anchor 620 illustrated in FIG. 9A and FIG. 9B as compared with the example of an embodiment of an anchor 220 in FIG. 5A and FIG. 5B, may result in a greater talon base region 622b and/or less space or distance between the talons 620 than compared with a lesser degree of tapering as in the anchor 220 illustrated in FIG. 5A and FIG. 5B. Similarly, a smaller overall size (e.g., diameter) of an anchor, as in the example of an embodiment of an anchor 220 illustrated in FIG. 5A and FIG. 5B as compared with the example of an embodiment of an anchor 720 in FIG. 10A and FIG. 10B, may result in less space or distance between the talons 220 than compared with a larger anchor as the anchor 720 illustrated in FIG. 10A and FIG. 10B.

It will be appreciated that in the illustrated examples of embodiments described above, the talons are generally evenly spaced apart. However, the talons need not be evenly spaced apart in other embodiments (not illustrated). Moreover, it will be appreciated that the talons of an anchor may have different sizes, shapes, configurations, and/or dimensions. The thickness of the material of the anchor talons (in a radial direction such as between the interior surface and the exterior surface of the talon, or the interior and exterior of the anchor) may be selected to allow automatic shifting or bending back of the talons while also not being susceptible to breakage. Additionally or alternatively, the width of the talons (along the perimeter or circumference of the anchor) may be selected to allow automatic shifting or bending back of the talons while also not being susceptible to breakage.

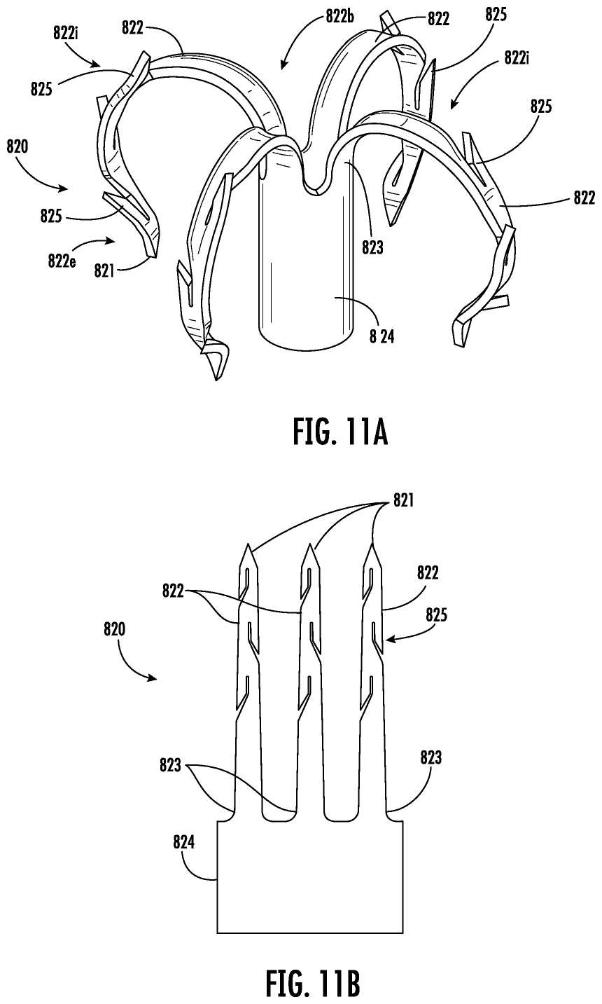

The shape and dimensions of talons of anchors formed in accordance with various principles of the present disclosure may be selected to enhance purchase of the anchor talons with tissue to which the anchor is secured. Additionally or alternatively, the shapes and dimensions of anchor talons formed in accordance with various principles of the present disclosure may be selected to enhance purchase of the anchor talons with tissue to which the anchor is secured. In some embodiments, additional features may be provided on the talons to increase purchase with tissue. For example, in the example of an embodiment of an anchor 820 illustrated in FIG. 11A and FIG. 11B, one or more barbs 825 may be formed along one or both sides of one or more of the anchor talons 822. The barbs 825 may be formed in any desired manner, such as by forming cuts in the talons 822 and bending (and optionally also heat setting) a small portion of the talon 822 along the cut away from the remaining portion of the talon 822. The barbs 825 may be sized, shaped, configured, and dimensioned to effectively increase the width of the talons 822 to provide a wider footprint within the tissue in which the talons 822 are inserted.

In embodiments in which the anchor is anchored in heart tissue (such as to anchor an artificial chordae tendineae to papillary muscle), the strength of the talons generally must be balanced with the flexibility of the talons. For instance, the talons should be sufficiently strong to endure multiple palpatory forces thereon (e.g., over 800 million cycles), yet not to deform or break upon changing shape (e.g., bending or straightening, such as when shifting between the delivery and deployed configurations). The thickness (in a radial direction) of at least some of the talons may be modified to achieve the desired pull out resistance/purchase strength in tissue (thicker talons generally are stronger with a greater holding force) while not being so thick as to potentially deform or break. Additionally or alternatively, the width of the talons may be selected to achieve the desired tissue purchase strength as well as material strength (to resist deforming or breaking). The width of the talons may vary, such as described above. A generally continuous taper from the base of the talon to the distal end of the talon, as illustrated in FIG. 6A and FIG. 6B, and optionally a generally wider width with less space between talons, as illustrated in FIG. 9A and FIG. 9B, may provide the desired strength at the base and/or to distribute stress and/or strain on the talons upon bending, and/or the desired characteristics for an anchor for an artificial chordae tendineae 150. Optionally, talons with two tapers, as illustrated in FIG. 7A, FIG. 7B, FIG. 8A, FIG. 8B, may provide increased strength to the talons 422, 522 along the intermediate regions 422i, 522i thereof. In some embodiments, the talons may have a thickness which is less than the width of the talons. In some embodiments, the cross-sectional shape of the talons may be substantially rectangular. The length of the tube and/or talons formed therefrom may be selected so that the talons penetrate sufficiently into tissue, yet are not so long so that the distal ends thereof exit the tissue (e.g., enter from one side and exit from another side, or enter and bend through the tissue and exit from the same side of the tissue as the point of entry). In some embodiments, the talons are laser cut from a tube with a thickness of approximately 0.0075 in (0.1905 mm) and the thickness of at least some of the talons (and optionally the anchor body as well) is reduced (e.g., by electropolishing) to approximately 0.0055 in (0.1397 cm) or less.

Anchor talons formed in accordance with various principles of the present disclosure may be formed from an anchor base forming the anchor body or may be coupled to a separately formed anchor body. In some embodiments, anchor talons are formed integrally with the anchor base/anchor body. In some embodiments, an anchor is formed in accordance with various principles of the present disclosure from a tube, the talons being formed by cutting the tube into the desired number and shapes of talons. For example, an anchor may be formed from a laser-cut tube, such as a nitinol tube. Planar projections showing examples of cutting patterns forming various configurations of anchors 220, 320, 420, 520, 620, 720, 820 (respectively illustrated in FIG. 5A, FIG. 6A, FIG. 7A, FIG. 8A, FIG. 9A, FIG. 10A, and FIG. 11A) are illustrated in FIG. 5B, FIG. 6B, FIG. 7B, FIG. 8B, FIG. 9B, FIG. 10B, and FIG. 11B. Once the tube has been cut, the talons may be shaped (e.g., heat set, and typically in a series of steps to gradually increase the bend in the talons) into the desired configuration for engaging and anchoring into tissue. The tube may be further modified, such as electropolished, to form a tissue anchor in accordance with various principles of the present disclosure.

As described above with respect to FIG. 3 and FIG. 4, the anchor base or the anchor body may be coupled with an artificial chordae tendineae tensioning and locking device 140. In some embodiments, the anchor body may be provided with a bore through which a pin may extend to couple the anchor body with an artificial chordae tendineae tensioning and locking device 140. Examples of bores 245, 345, 445, 545, 645, 745, 845, through the anchor body 224, 324, 424, 524, 624, 724, 824 of respective anchor 220, 320, 420, 520, 620, 720, 820 are illustrated in FIG. 5A, FIG. 5B, FIG. 6A, FIG. 6B, FIG. 7A, FIG. 7B, FIG. 8A, FIG. 8B,

FIG. 9A, FIG. 9B, FIG. 10A, FIG. 10B, FIG. 11A, FIG. 11B. It will be appreciated the bores for coupling components of anchor delivery and deployment system 100 formed in accordance with principles of the present disclosure may be any shape acceptable to one of ordinary skill in the art.

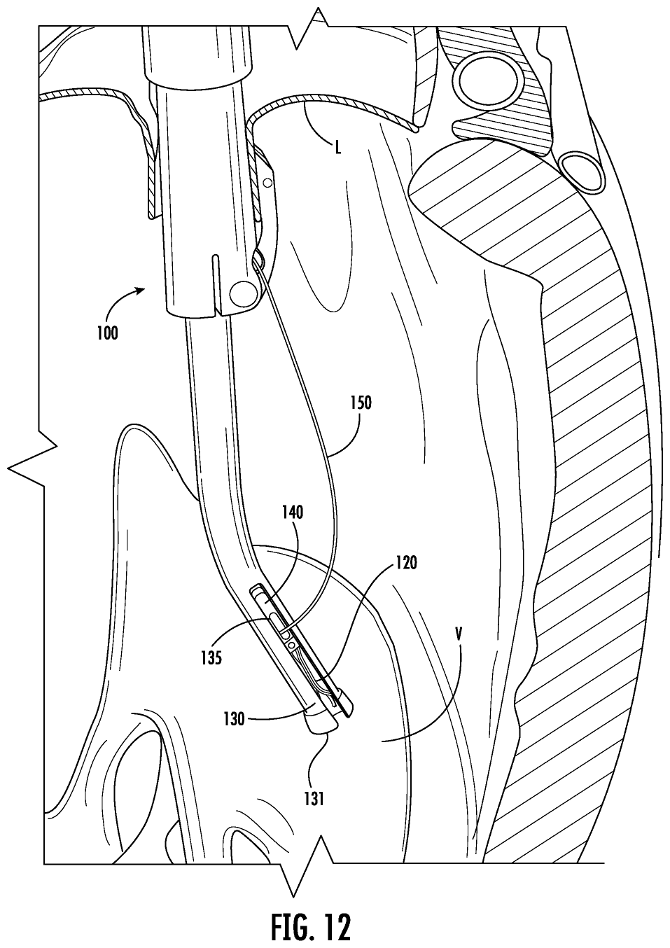

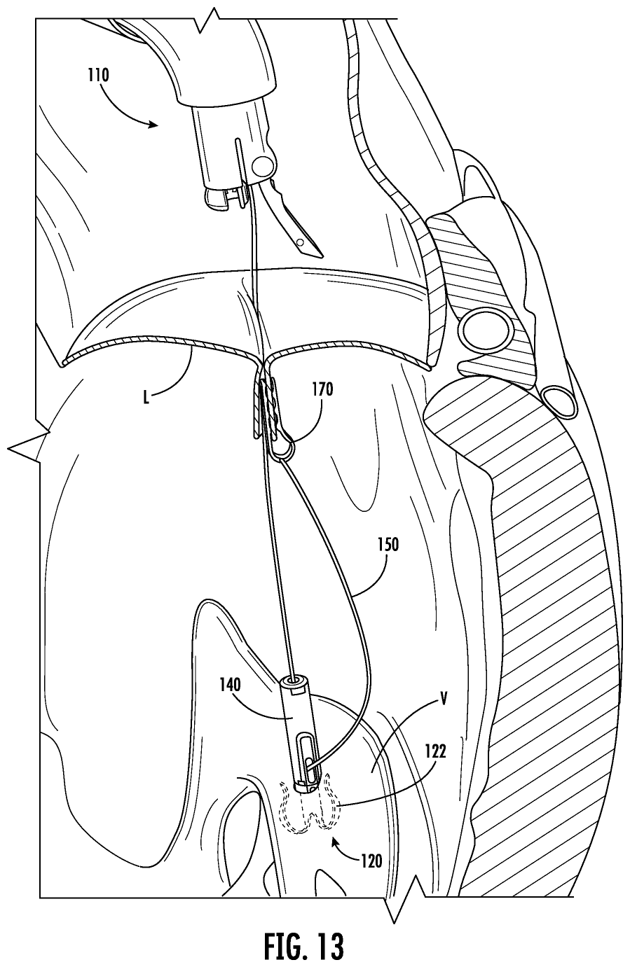

In use, an anchor delivery and deployment system 100 formed in accordance with various principles of the present disclosure may be used to deliver and deploy an anchor 120 (optionally considered a part of the anchor delivery and deployment system 100) to heart tissue. In the example of an embodiment illustrated in FIG. 12 and FIG. 13, an artificial chordae tendineae 150 is clamped to a heart valve leaflet L, such as with a leaflet clip 170 delivered and deployed with a leaflet clip delivery and deployment system such as disclosed in above-referenced and above-incorporated U.S. Patent Application Publication US2021/0000597 or U.S. Patent Application Publication US2022/0096235. The leaflet clip delivery and deployment system may be delivered with and optionally be considered a part of an anchor delivery and deployment system 100 formed in accordance with various principles of the present disclosure. For instance, a leaflet clip delivery and deployment system may be mounted over the anchor garage 130. A leaflet clip spreader may be used to deploy a leaflet clip, and may be detached from the leaflet clip once an anchor 120 has been deployed, and then may be removed from the delivery/deployment site. The artificial chordae tendineae 150 may extend from the leaflet clip 170 be coupled to an anchor 120 formed in accordance with various principles of the present disclosure. For instance, the artificial chordae tendineae 150 may extend into the anchor garage 130 to be coupled with an artificial chordae tendineae tensioning and locking device 140 with which the anchor 120 is coupled. As the anchor delivery and deployment system 100 is advanced to deliver the anchor 120 to heart tissue (such as the papillary muscle tissue of a heart valve HV), the artificial chordae tendineae 150 is advanced to the heart tissue as well, as illustrated in FIG. 12. The distal end 131 of the anchor garage 130 is pressed against the heart tissue at the deployment site for the anchor 120, as illustrated in FIG. 1, and the anchor pusher 160 (see, e.g., FIG. 3 and FIG. 4) may be advanced distally to the deployment site to push the anchor 120 out of the anchor garage 130 and into the heart tissue. Once the talons 122 of the anchor 120 are extended out from within the anchor garage 130, and into the heart tissue, the talons 122 shift to an extended configuration such as illustrated in phantom in FIG. 13. The anchor delivery and deployment system 100 may be retracted (moved proximally), such as illustrated in FIG. 13, leaving the anchor 120 and the artificial chordae tendineae 150 secured to heart tissue. The artificial chordae tendineae tensioning and locking device 140 may be adjusted to adjust tension on the artificial chordae tendineae 150. The artificial chordae tendineae 150 may be cut to a desired length and left implanted with the leaflet clip 170 and the anchor 120.

In view of the above, it should be understood that the various embodiments illustrated in the figures have several separate and independent features, which each, at least alone, has unique benefits which are desirable for, yet not critical to, the presently disclosed tissue anchors and anchor delivery and deployment devices, systems, and methods. Therefore, the various separate features described herein need not all be present in order to achieve at least some of the desired characteristics and/or benefits described herein. Only one of the various features described herein may be present in a tissue anchor or anchor delivery and deployment system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An anchor delivery and deployment system comprising:
an anchor having an anchor body and a plurality of anchor talons extending from the anchor body and shiftable between a delivery configuration and a deployed configuration; and
an anchor garage configured to house the anchor therein with the anchor talons in a delivery configuration, and having an open blunt end through which the anchor is deployable;
wherein the anchor talons expand from the delivery configuration when within the anchor garage to the deployment configuration when deployed outside the anchor garage.

2. The delivery and deployment system of claim 1, wherein the blunt end of the anchor garage has a convex curved outer surface to present a blunt end for pressing against tissue without damaging the tissue.

3. The delivery and deployment system of claim 1, wherein:
the anchor talons extend from a talon base coupled to the anchor body to a talon distal end configured to penetrate tissue; and
at least one of the talons is tapered between the talon base and the talon distal end.

4. The delivery and deployment system of claim 3, wherein at least one of the talons is tapered toward the talon distal end.

5. The delivery and deployment system of claim 4, wherein at least one of the talons is tapered from the talon base to the talon distal end.

6. The delivery and deployment system of claim 4, wherein at least one of the talons has an intermediate region between the talon base and the talon distal end, the at least one of the talons being tapered from the intermediate region to the talon distal end.

7. The delivery and deployment system of claim 6, wherein the at least one of the talons is tapered from the intermediate region to the talon base.

8. The delivery and deployment system of claim 3, wherein the at least one of the talons is tapered widthwise.

9. The delivery and deployment system of claim 8, wherein the anchor is formed from a tube, the talons being cut from the tube.

10. The delivery and deployment system of claim 1, wherein the anchor is formed from a tube, the talons being cut from the tube.

11. The delivery and deployment system of claim 1, further comprising an artificial chordae tendineae coupled to the anchor.

12. A tissue anchor comprising:
an anchor body formed from a tube; and
a plurality of anchor talons formed by cutting the tube;
wherein:
the anchor talons extend from a talon base coupled to the anchor body to a talon distal end configured to penetrate tissue; and
at least one of the talons is tapered between the talon base and the talon distal end.

13. The tissue anchor of claim 12, wherein at least one of the talons is tapered toward the talon distal end.

14. The tissue anchor of claim 13, wherein at least one of the talons is tapered from the talon base to the talon distal end.

15. The tissue anchor of claim 13, wherein at least one of the talons has an intermediate region between the talon base and the talon distal end, the at least one of the talons being tapered from the intermediate region to the talon distal end.

16. The tissue anchor of claim 12, wherein the at least one of the talons is tapered from the intermediate region to the talon base.

17. The tissue anchor of claim 12, wherein the at least one of the talons is tapered widthwise.

18. The tissue anchor of claim 12, wherein the talons are formed by laser cutting the tube from which the anchor body is formed.

19. A method of securing an anchor in tissue, the method comprising:
delivering a tissue anchor in an anchor garage, the anchor garage having an open blunt distal end, the tissue anchor having a plurality of talons each extending from a talon base to a talon distal end;
pressing the blunt distal end of the anchor garage against tissue without damaging the tissue;
advancing the anchor talon distal ends out of the blunt distal end of the anchor garage and to penetrate tissue; and
advancing the anchor out of the anchor garage to deploy the anchor in tissue, allowing the anchor talons to shift from a delivery configuration to a deployed configuration within the tissue.

20. The method of claim 19, wherein the anchor and anchor talons are formed from a laser cut tube.
